(19) United States
(12) Patent Application Publication
(10) Pub. No.: US 2011/0029018 A1
(43) Pub. Date: Feb. 3, 2011

(54) VARIABLE RESISTANCE SPINAL STABLIZATION SYSTEMS AND METHODS

(75) Inventor: Gil Carlos, Collierville, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Othropedic, Inc, Warsaw, IN (US)

(21) Appl. No.: 12/512,151

(22) Filed: Jul. 30, 2009

(51) Int. Cl. A61B 17/70; A61B 17/88

(52) U.S. Cl. 606/246; 606/279

(57) ABSTRACT

Systems, devices and methods for stabilizing a portion of the spinal column using one or more elongate members anchored to a number of vertebrae. The elongate member includes a portion configured to prevent, or at least substantially resist, lateral bending motion of one or more vertebral levels while allowing flexion and extension motion of the one or more vertebral levels.

### BACKGROUND

The spine is subject to various pathologies that compromise its load bearing and support capabilities. Such pathologies of the spine include, for example, degenerative diseases, the effects of tumors, and fractures and dislocations attributable to physical trauma. In the treatment of diseases, malformations or injuries affecting one or more spinal motion segments (which include two or more adjacent vertebrae and the disc tissue or disc space therebetween), and especially those affecting disc tissue, removal of some or all of a degenerated, ruptured or otherwise failing disc is sometimes required. It is also known that artificial discs, fusion implants, or other interbody devices can be placed into the disc space subsequent to removal of disc material. External stabilization of the spinal motion segments, alone or in combination with interbody devices, may be accomplished via attachment of one or more elongate plates, rods or other external stabilization devices to the spinal column.

Additionally, current operative methods for treating spinal deformities, particularly scoliosis, include correction of the spinal curvature via some form of internal fixation device, and fusion of the spine in the corrected state may be accomplished by the placement of bone graft between the adjacent vertebrae. Several instrumentation systems are available to correct and stabilize the spinal column while fusion occurs. Nonoperative methods also exist and may be used when applicable. These nonoperative methods include, for example, bracing and observation.

Patients with infantile or juvenile scoliosis who undergo curve stabilization via the use of subcutaneous rods are subject to multiple surgical procedures for lengthening and adjustment of the rods as the patient grows. Moreover, rigid stabilization systems that substantially prevent flexion and extension motion of the vertebrae can loosen bone screws to which the rod is attached, requiring surgical procedures to revise the bone screws. As should be appreciated, it is generally preferable that the number of surgical procedures required for treatment of the spinal column be minimized. Additionally, anterior or posterior spinal fusion in a skeletally immature patient often results in loss of vertebral body height and girth. Another problem that sometimes arises is that some children are not physically able to tolerate the surgical procedures required for a definitive fusion procedure. Further, poor self-image may occur in adolescent patients who are externally braced for scoliosis.

While prior spinal stabilization and support systems are a step in the right direction, there remains room for additional improvements. For example, subsequent to implantation of prior stabilization systems, additional surgeries are sometimes required in order to adjust one or more components associated with the spinal construct, such as lengthening of a spinal rod of the construct or to revise the anchors which secure the spinal construct to the vertebrae.

Thus, there is a general need in the industry to provided improved systems, devices and methods for stabilizing a portion of the spinal column using one or more elongate members. There is also a need to provide improved systems, devices and methods that reduce the number and/or frequency of adjustments to accommodate for continued growth of the patient's spinal column, particularly in cases involving adolescents or young adults having an immature spine.

### SUMMARY

The present invention relates generally to systems, devices and methods for stabilizing a portion of the spinal column using one or more elongate members anchored to a number of vertebrae. The elongate member includes a portion configured to prevent, or at least substantially resist, lateral bending motion of one or more vertebral levels while allowing flexion and extension motion of the one or more vertebral levels.

According to one aspect, a spinal stabilization system comprises at least one bone anchor; and an elongate member engageable to the at least one bone anchor when the elongate member is positioned along a spinal column of a patient with a longitudinal axis of the elongate member oriented in a caudal to cephalad direction relative to the patient. The elongate member includes an elongate caudal portion extending from a caudal end of the elongate member and an elongate cephalad portion extending from a cephalad end of the elongate member toward the caudal portion. The elongate member further includes an elongate transition portion extending between and connecting the caudal portion to the cephalad portion. The caudal portion includes a cross-section orthogonal to the longitudinal axis that is elliptical and the cephalad portion includes a cross-section orthogonal to a center axis of the cephalad portion that is circular. The transition portion includes a cross-section orthogonal to a center axis of the transition portion that progressively transitions from the elliptical cross-section of the caudal portion at a first junction with the caudal portion to the circular cross-section at a second junction with the cephalad portion.

According to another aspect, a spinal stabilization system comprises at least one bone anchor and an elongate member engageable to the at least one bone anchor when the elongate member is positioned along a spinal column of a patient with a longitudinal axis of the elongate member oriented in a caudal to cephalad direction relative to the patient. The elongate member includes an elongate caudal portion extending from a caudal end of the elongate member and an elongate cephalad portion extending from a cephalad end of the elongate member toward the caudal portion. The elongate member further includes an elongate transition portion extending between and connecting the caudal portion to the cephalad portion. The caudal portion includes a cross-section orthogonal to the longitudinal axis that is substantially stiffer between medial and lateral sides of the caudal portion than between anterior and posterior sides of the caudal portion. The cephalad portion includes a cross-section orthogonal to a center axis of the cephalad portion that provides substantially the same stiffness in all directions from the center axis of the cephalad portion. The transition portion includes a cross-section orthogonal to a center axis of the transition portion that gradually transitions from the cross-section of the caudal portion at a first junction with the caudal portion to the cross-section of the cephalad portion at a second junction with the cephalad portion.

According to another aspect, a method for spinal stabilization comprises: providing an elongate member for positioning along a plurality of vertebral levels of a spinal column of a patient, wherein the elongate member includes an elongate caudal portion extending from a caudal end toward a cephalad end of the elongate member, the elongate member further including a cephalad portion extending from the cephalad end toward the caudal end, and a transition portion extending from the caudal portion to the cephalad portion; positioning the caudal portion along at least two vertebral levels of the plurality of vertebral levels with a major dimension of an elliptical cross-section of the caudal portion oriented in a medial-lateral direction to prevent lateral bending of the at least two vertebral levels and a minor dimension of the elliptical cross-section of the caudal portion oriented in the anterior-posterior direction to permit flexion and extension movement of the at least two vertebral levels along which the caudal portion extends; positioning the cephalad portion along at least one vertebral level of the plurality of vertebral levels, wherein the cephalad portion includes a circular cross-section; positioning the transition portion along at least two vertebral levels of the plurality of vertebral levels, wherein the transition portion includes a cross-section that gradually transitions from the elliptical cross-section of the caudal portion to the circular cross-section of the cephalad portion; and anchoring the cephalad portion to at least one vertebra of the spinal column.

In one refinement of the method, there is provided a second elongate member extending caudally from the caudal end of the caudal portion and the second elongate member is anchored to at least one vertebra of the spinal column. The caudal portion and the transition portion span the vertebral levels between the anchoring of the second elongate member and the cephalad portion without being anchored to any of the vertebrae between the vertebrae to which the cephalad portion and second elongate member are anchored.

Further embodiments, forms, features, aspects, benefits, objects and advantages of the present invention shall become apparent from the detailed description and figures provided herewith.

### BRIEF DESCRIPTION OF THE FIGURES

FIG. 1 is a posterior view of a spinal stabilization system according to one form of the present invention, as attached to a posterior aspect of the spinal column.

FIG. 2 is a side view of a portion of the elongate member of the spinal stabilization system of FIG. 1.

FIG. 2A is an enlarged view of the connection between first and second portions of the elongate member.

FIG. 3 is a section view along line 3-3 of FIG. 2.

FIG. 4 is a section view along line 4-4 of FIG. 2.

FIG. 5 is a section view along line 5-5 of FIG. 2.

FIG. 6 is a section view along line 6-6 of FIG. 2.

FIG. 7 is a section view along line 7-7 of FIG. 2.

FIG. 8 is a section view along line 8-8 of FIG. 2.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation on the scope of the invention is intended. Any alterations and further modifications in the illustrated devices and described methods and further applications of the principles of the invention as disclosed herein are contemplated as would normally occur to one skilled in the art to which the invention relates.

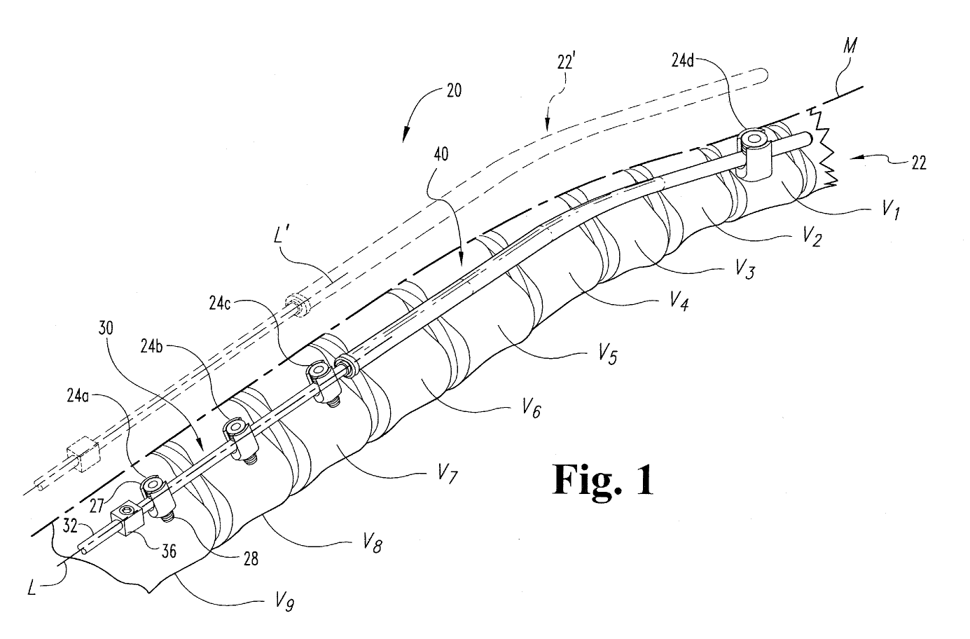

Referring to FIG. 1, shown therein is a stabilization system 20 for stabilizing at least a portion of the spinal column. In the illustrated embodiment, the stabilization system 20 extends across a plurality of vertebral levels including vertebrae V₁-V₉. However, it should be understood that the stabilization system 20 may extend across any number of vertebral levels including two or more vertebrae. Additionally, in the illustrated embodiment, the stabilization system 20 is attached to a posterior aspect of the spine. However, it should also be understood that the stabilization system 20 may be attached to other aspects of the spine, including anterior, antero-lateral, lateral, and/or postero-lateral aspects of the spine. Further, although the stabilization system 20 is contemplated for use in association with the thoracic region of the spine, it should be understood that the stabilization system 20 may be utilized in other regions of the spine, including the cervical, lumbar, lumbo-sacral and sacral regions, and combinations or two or more of these regions, of the spine. It should also be understood that the stabilization system 20 can be used in association with fusion or fusionless treatment of the spine.

In the illustrated embodiment of FIG. 1, vertebrae V₁-V₉ are shown diagrammatically for purposes of clarity, and only the right lateral half of each vertebra is shown along one side of spinal midline M. It should be understood that other lateral halves of the V₁-V₉ not shown may also include an elongate member 22′ (shown in phantom) attached to one or more the vertebrae V₁-V₉ so that a pair of elongate members 22, 22′, each extending along a respective longitudinal axis L, L′, are engaged across a number of vertebral levels via a plurality of anchor members 24. However, it should be understood that stabilization system 20 may utilize any number of elongate members 22, including a single elongate member or three or more elongate members. In the illustrated embodiment, elongate member 22 includes a first portion 30 engaged to the spinal column by anchor members 24a, 24b, 24c at a location at or near the caudal end of the elongate member 22, and a second portion 40 that is removably engageable to first portion 30 engaged by a fourth anchor 24d to vertebra V1 at or near the cephalad end of elongate member 22. In one specific technique, fourth anchor 24d is engaged one or more levels above (cephalad to) the coronal apex of the thoracic curve and anchors 24a-c are engaged to vertebrae one or more levels below the coronal apex. Engagement of one or more elongate members 22 to the spine in this manner is sometimes referred to as the Shilla technique. Although elongate member 22 is specifically illustrated as being specifically engaged to the vertebrae V₁, V₇, V₈ and V₉, it should be understood that other embodiments of the systems and techniques discussed herein contemplate that elongate members 22, 22′ may be engaged to each of the vertebrae V₁-V₉, to every other one of the vertebrae V₁-V₉, or to any number of the vertebrae V₁-V₉ via other layouts or attachment configurations and techniques.

According to one aspect of stabilization system 20, the stabilization system 20 is configured to provide lateral stabilization or support to the portion of the spine being treated to substantially resist lateral bending of a portion of the stabilized vertebrae in the coronal plane, while at the same time allowing for at least some degree of relative movement or motion in flexion and extension in the sagittal plane of the portion of the vertebrae to which elongate member 22 is engaged. In one embodiment, engagement between elongate member 22 and at least one of the anchor members 24a-c is fixed or constrained so as to substantially prevent relative axial movement therebetween, and the engagement between elongate member 22 and anchor member 24d is variable or unconstrained so as to allow for relative axial movement therebetween. The anchor member 24d variably engaged to the elongate member 22 allows for sliding movement of the anchor member 24d along an axial length of elongate member 22 (e.g., in a direction generally along the sagittal and coronal planes), thereby allowing for relative axial movement or motion between the vertebrae to accommodate for continued growth of the patient's spine. Examples of suitable anchor 24d configurations are provided in U.S. Patent App. Pub. No. 2006/0241594, which is incorporated herein by reference in its entirety. In another embodiment, engagement between elongate member 22 and each of the anchor members 24a-d is fixed or constrained so as to substantially prevent relative axial movement therebetween.

In one embodiment, each of the first and second portions 30, 40 of elongate member 22 is generally configured as an elongate spinal rod. In the illustrated embodiment, first portion 30 includes a generally circular outer cross section; however, other suitable cross sections for first portion 30 are also contemplated including, for example, elliptical, triangular, rectangular, hexagonal or polygonal shapes and configurations, or any other suitable shape or configuration. In another embodiment, first portion 30 has a rigid or semi-rigid configuration suitable for providing a degree of lateral stabilization or support to prevent motion of the portion of the spinal column to which first portion 30 is attached. However, in other embodiments, first portion 30 may have a flexible or semi-flexible configuration and may exhibit resilient or semi-resilient characteristics. In addition, first portion 30 may be comprised of multiple rod components 32 and 34 connected to one another view a coupling member 36. Coupling member 36 provides a connection between rod components 32, 34 that have different sizes, cross-sectional shapes and/or material properties. Examples of suitable coupling members 36 are described in U.S. Patent Application Pub. Nos. 2005/0277932 and 2005/0277925, each of which is incorporated herein by reference in its entirety.

In one embodiment, elongate member 22 is formed of a metallic material such as, for example, titanium, a titanium alloy, stainless steel, a chrome-cobalt alloy, a shape-memory or superelastic material such as nitinol, or other suitable metallic materials known to those of skill in the art. In other embodiments, elongate member 22 may be formed of a polymer such as, for example, polyester, polyethylene, PEEK or from a synthetic material. It is further contemplated that the first and second portions 30, 40, and that the rod components 32, 34, can be formed of the same material or of differing materials.

In the illustrated embodiment, anchor members 24 are configured as bone anchors comprising a bone engaging portion 28 structurally configured for engagement with vertebral bone, and a receiver portion 27 structurally configured for engagement with one of the elongate members 22. Various types and configurations of anchor members are also contemplated as falling within the scope of the present invention including, for example, spinal screws, pedicle screws, multi-axial spinal screws, uni-axial spinal screws, spinal hooks, staples, various types and configurations of connectors, or other types of anchor members known to those of skill in the art that are suitable for engaging one or more elongate members 22 to the spinal column. In one embodiment, anchor members 24 are formed of a metallic material such as, for example, titanium, a titanium alloy, stainless steel, a chrome-cobalt alloy, a shape-memory or superelastic material such as nitinol, or other suitable metallic materials known to those of skill in the art. In other embodiments, anchor members 24 may be formed of a polymer such as, for example, polyester or polyethylene, or from a synthetic material. As discussed above, one or more of the anchors 24 may be of the constrained type to substantially prevent axial movement of the anchor relative to elongate member 22, and one or more of the anchors 24 may be of the unconstrained type to permit axial movement of the anchor relative to elongate member 22.

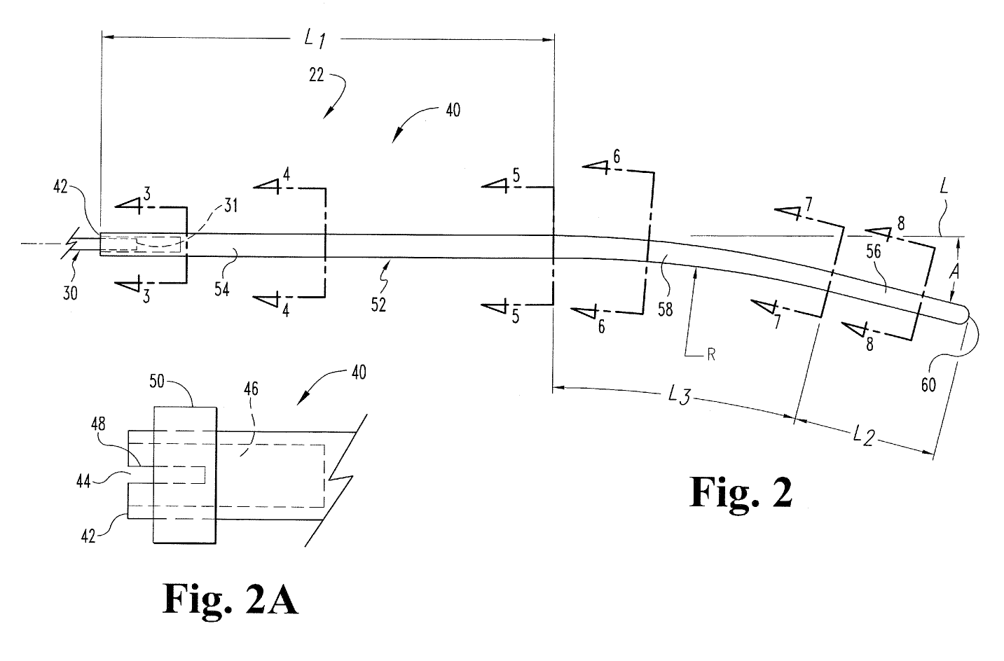

Referring now to FIGS. 2 and 2A, there is shown a side elevation view of elongate member 22 with second portion 40 and a part of first portion 30. The caudal end 42 of second portion 40 includes an axial end opening 44 forming a channel 46 that extends along a portion of the length of second end portion 40. The cephalad end 31 of second portion 30 is movably received through opening 44 and in channel 46 to allow the overall length of elongate member 22 to be adjusted to provide a desired fit with the spinal column, or to allow length adjustment post-implantation to accommodate growth of the patient. As shown in FIG. 2A, second portion 40 includes at least one slot 48 that extends through second portion 40 and opens into channel 46. Slot 48 allows flexing of caudal end 42 so that securing member 50 can clampingly engage first portion 30 to second portion 40 in channel 46. In one embodiment, securing member 50 is a ring that encircles the outer surface of second portion 40. The ring is movable along and clampable against second portion 40 to force second portion 40 against first portion 30 in channel 46, securing first and second portions 30, 40 to one another in end-to-end engagement.

In one particular embodiment, securing member 50 is at least partially formed of a shape-memory material that exhibits pseudoelastic or superelastic characteristics or behavior at about human body temperature. While the entire securing member 50 is formed of the shape-memory material in one embodiment, it should be understood that securing member 50 may also be formed using any suitable biocompatible material, such as, for example, stainless steel or titanium. There is a wide variety of shape-memory materials suitable for use with securing member 50, including shape-memory metal alloys (e.g., alloys of known metals, such as, for example, copper and zinc, nickel and titanium, and silver and cadmium) and shape-memory polymers. While there are many alloys which exhibit shape-memory characteristics, one of the more common SMAs is an alloy of nickel and titanium. One such alloy is nitinol, which is a bio-compatible SMA formed of nickel and titanium. Nitinol is well suited for the particular application of securing member 50 because it can be programmed to undergo a stress-induced martensitic transformation at about normal human body temperature (i.e., at about 35-40 degrees Celsius). Thus, when implanted securing member 50 returns toward its memorized configuration, which can be sized to cause securing member 50 to compress second portion 40 and secure it to first portion 30. It should be understood, however, that other SMA materials that exhibit superelastic characteristics are contemplated.

Referring FIG. 2, additional features of second portion 40 of elongate member 22 will be discussed further. Second portion 40 includes an elongate body 52 having a caudal portion 54 and a cephalad portion 56. In one embodiment, a curved transition portion 58 extends between and connects portions 54, 56 to one another. In this embodiment, caudal portion 54 extends along and is centered on longitudinal axis L, while cephalad portion 56 diverges from longitudinal axis L at an angle A so that cephalad end 60 is offset from longitudinal axis L. When implanted as shown in FIG. 1, cephalad end 60 is offset in the anterior direction relative to the caudal end 42. In one embodiment, transition portion 58 extends along an arc defined by a radius R. Radius R in one specific embodiment is about 11.7 inches, although other embodiments contemplate other dimensions for the radius R.

In another embodiment, transition portion 58 and cephalad portion 56 are aligned with and centered on longitudinal axis at least prior to implantation. The surgeon can bend transition portion 58 to provide a desired fit with the anatomy prior to implantation or in situ. In yet another embodiment, transition portion 58 is flexible so that it bends and conforms to the patient anatomy when it is secured to the anchors.

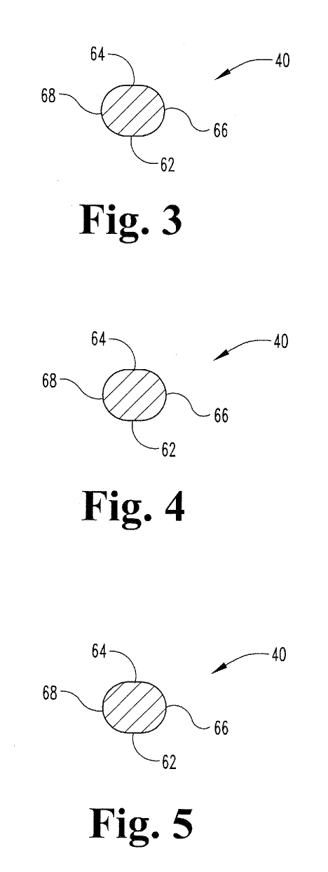

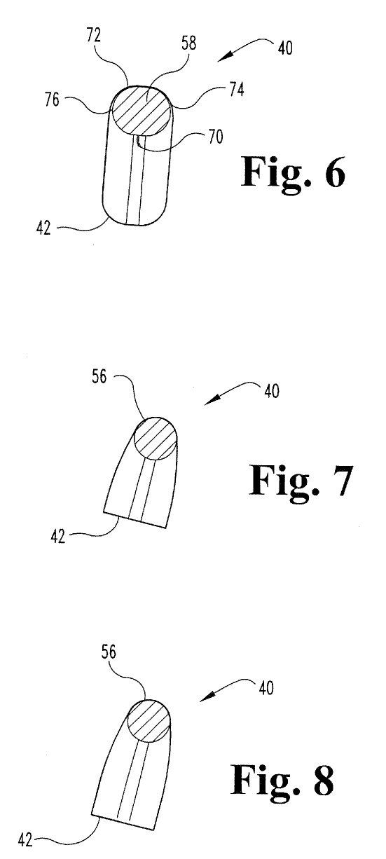

Caudal portion 54 includes a cross-sectional shape that provides substantial resistance to lateral bending in the coronal plane of the vertebrae along which it extends, while allowing flexion and extension movement of the vertebrae in the sagittal plane. As shown in FIGS. 3-5, caudal portion 54 includes an elliptical or oval cross-section. The cross-section includes anterior and posterior sides 62, 64 that are linear and extend between rounded medial and lateral sides 66, 68. In one particular embodiment, caudal portion 54 includes a height between anterior and posterior sides 62, 64 of about 0.236 inches, and a width between medial and lateral sides 66, 68 of about 0.295 inches. Medial and lateral sides 66, 68 are curved between anterior and posterior sides 62, 64 along an arc having a radius of about 0.118 inches. The elliptical cross-section extends along substantially all the length of caudal portion 54 from caudal end 42 to transition portion 58. In one specific embodiment, the overall length L1 of caudal portion 54 from caudal end 42 to transition portion 58 is about 5 inches. It should be understood, however, that other embodiments contemplate other cross-sectional dimensions and lengths for caudal portion 54.

Cephalad portion 56 includes a linear configuration extending cephaladly from transition portion 58 and diverging away longitudinal axis L to cephalad end 60. As shown in FIGS. 7 and 8, cephalad portion includes a circular cross-section. In one embodiment, the circular cross-section has a diameter of about 0.197 inches, and cephalad portion 56 includes a length L2 of about 1.5 inches from transition portion 58 to cephalad end 60. It should be understood, however, that other embodiments contemplate other cross-sectional dimensions and lengths for cephalad portion 56.

Transition portion 58 includes a longitudinally varying cross-section that extends along an arc from caudal portion 54 to cephalad portion 56. The cross-section of transition portion 58 gradually transitions from the elliptical cross-section of caudal portion 54 where transition portion 58 joins caudal portion 54 to the circular cross-section of cephalad portion 56 where transition portion 58 joins cephalad portion 56. Accordingly, transition portion 58 includes linear anterior and posterior sides 70, 72 connected to anterior and posterior sides 62, 64, respectively, at its junction with caudal portion 54 that extend to cephalad portion 56. Each of the anterior and posterior sides 70, 72 longitudinally transitions in width so that medial and lateral sides 74, 76 of transition portion 58 converge toward one another in a direction to a point at the junction of transition portion 58 with cephalad portion 56. Anterior and posterior sides 70, 72 of transition portion 58 each longitudinally transition to a point having no width at its junction with cephalad portion 56. Furthermore, each of medial and lateral sides 74, 76 has a first radius that is the same radius as medial and lateral sides 66, 68 at the junction of transition portion 58 with caudal portion 54. Each of medial and lateral sides 74, 76 has a longitudinally variable radius that transitions from the first radius to a second radius, and the second radius is the same as the radius of the circular cross-section of cephalad portion 56. In one specific embodiment, the radius of each of the medial and lateral sides 74, 76 of transition portion 58 gradually transitions from 0.236 inches at the junction with caudal portion 54 to 0.197 inches at the junction with cephalad portion 56. It should be understood, however, that other embodiments contemplate other cross-sectional dimensions and lengths for transition portion 58.

Portion 40 of elongate member 22 is a stabilization device with stiffness profiles that provided varying degrees of resistance to bending about its center axis along the length of portion 40. Caudal portion 54 has a stiffness across the major dimension of its elliptical cross-section that is substantially greater than the stiffness across the minor dimension of the elliptical cross-section to resist lateral bending motion of the vertebral levels along which caudal portion extends while allowing at least limited flexion and extension motion. Cephalad portion 56 provides has a stiffness profile that is the same in all directions from its center axis to provide the same resistance to lateral bending movement and flexion/extension movement. Transition portion 58 provides a gradual transition of the greater resistance to lateral bending afforded by caudal portion 54 to the lesser resistance afforded by cephalad portion 56. This gradual transition in resistance extends along at least one vertebral level, and preferably at least two vertebral levels, so that degree of resistance to lateral bending is not abruptly changed from one vertebral level to the next vertebral level. In one specific embodiment, caudal portion 54 has a length corresponding to about 50% of the overall length of portion 40 of elongate member 22, and cephalad portion 56 has a length corresponding to about 15% of the overall length of portion 40 of elongate member 22. In yet another embodiment, the length L1 of caudal portion 54 is greater than the length L3 of transition portion 58, and length L3 is greater than length L2 of cephalad portion 56. In one specific embodiment, length L1 is about one half of the overall length of portion 40, and length L3 is about twice the length of L2.

Having described the various features associated with spinal stabilization system 20, reference will now be made to assembly of the stabilization system 20 and the interaction between elongate member 22, anchor members 24 and the spinal column of the patient according to one embodiment. As indicated above, in one embodiment, stabilization system 20 includes a pair of elongate spinal rods 22 engaged across a number of vertebral levels via a plurality of the anchor members 24. In the illustrated embodiment, anchor member 24 engaged to at least one the vertebrae is of the fixed or constrained type and anchor member 24 engaged to at least one of the other vertebra is of the variable or unconstrained type. However, as indicated above, it should be understood that the anchor members may be anchored to any of the vertebrae via other layout arrangements or configurations. For example, all of the anchors may be of the fixed type.

Once the anchor members 24 are properly anchored to the vertebrae, elongate member 22 is inserted into generally aligned passages of the anchors 24. In one embodiment, elongate member 22 includes portion 40 with cephalad portion 56 received in the anchor of the unconstrained type while caudal portion 54 and transition portion 58 are not received within or directly engaged to any of the vertebrae with any anchor. Rather, caudal portion 54 is removably engaged to another elongated rod portion 30 that extends caudally from caudal portion 54, and elongated rod portion 30 is engaged to one or more of the vertebrae with a constrained type of anchor 24. In this arrangement, the elliptical cross-section of caudal portion 54 is implanted with the linear anterior and posterior sides 62, 64 extending in the medial-lateral direction so that the orientation and sizing of the larger dimension of the elliptical cross-section is positioned to prevent or substantially resist lateral bending motion of the vertebral levels along which caudal portion 54 is engaged. The smaller anterior-posterior dimension of caudal portion 54 extending between anterior and posterior sides 62, 64 is implanted in alignment with the sagittal plane so that medial and lateral sides 66, 68 are oriented medially and laterally, the orientation and sizing of the smaller dimension being arranged to permit flexion and extension movement of the vertebral levels along which caudal portion 54 extends.

Following insertion of the elongate member 22 into the passages of the anchor members 24, a setscrew or other device is engaged to anchors 24 to secure elongate member 22 to anchors 24. In one embodiment, cephalad end 56 is positioned in an anchor 24 of the non-constrained type, permitting axial movement of the vertebra relative to cephalad portion 56 in response to, for example, growth of the patient, while providing at least some resistance to lateral bending and flexion/extension motion of the spinal column. In another embodiments, cephalad portion 56 is engaged to an anchor of a fixed type, and length adjustment is accomplished by loosening securing member 50 and axially repositioning first and second portions 30, 40 relative to one another. Combinations of these embodiments are also contemplated to provide flexibility to the surgeon in monitoring and selecting the desired length adjustment option to be used both during surgery and post-implantation.

In another embodiment technique, portion 40 comprises the entire length of elongate member 22 with caudal portion 54 and cephalad portion 56 each received in at least one anchor and transition portion 58 not received within or directly engaged to any of the vertebrae with any anchor. In this arrangement, the elliptical cross-section of caudal portion 54 is implanted with the linear anterior and posterior sides 62, 64 extending in the medial-lateral direction so that the orientation and sizing of the larger dimension of the elliptical cross-section is positioned to prevent or substantially resist lateral bending motion of the vertebral levels along which caudal portion 54 is engaged. The smaller anterior-posterior dimension of caudal portion 54 extending between anterior and posterior sides 62, 64 is implanted in alignment with the sagittal plane so that medial and lateral sides 66, 68 are oriented medially and laterally, the orientation and sizing of the smaller dimension being arranged to permit only flexion and extension movement of the vertebral levels along which caudal portion 54 extends.

As should be appreciated, the number and frequency of subsequent surgical procedures required for adjustment of stabilization system 20 to accommodate for growth of the patient's spinal column can be significantly reduced, if not eliminated. The increased medial-lateral dimension caudal portion 54 of the second portion 40 of elongate member 22 provides lateral stabilization and control in the coronal plane to prevent lateral bending motion of the vertebral levels along which caudal portion 54 extends while supporting the portion of the spinal column being treated. The reduced dimension in the anterior-posterior direction of the elongate member 22 aligned with the sagittal plane allows flexion and extension of the vertebrae in the sagittal plane, while reducing the forces exerted on the bone anchors and controlling growth of the spinal column.

Any theory, mechanism of operation, proof, or finding stated herein is meant to further enhance understanding of the present invention, and is not intended to make the present invention in any way dependent upon such theory, mechanism of operation, proof or finding. It should be understood that while the use of the word preferable, preferably or preferred in the description above indicates that the feature so described may be more desirable, it nonetheless may not be necessary, and embodiments lacking the same may be contemplated as within the scope of the application, that scope being defined by the claims that follow. In reading the claims, it is intended that when words such as “a”, “an”, “at least one”, and “at least a portion” are used, there is no intention to limit the claim to only one item unless specifically stated to the contrary in the claim. Further, when the language “at least a portion” and/or “a portion” is used, the item may include a portion and/or the entire item unless specifically stated to the contrary.

While the application has been illustrated and described in detail in the drawings and foregoing description, the same is to be considered as illustrative and not restrictive in character, it being understood that only the selected embodiments have been shown and described and that all changes, modifications and equivalents that come within the spirit of the invention as defined herein or by any of the following claims are desired to be protected.

What is claimed is:

1. A spinal stabilization system, comprising:
at least one bone anchor; and
an elongate member engageable to said at least one bone anchor when said elongate member is positioned along a spinal column of a patient with a longitudinal axis of said elongate member oriented in a caudal to cephalad direction relative to the patient, wherein said elongate member includes an elongate caudal portion extending from a caudal end of said elongate member and an elongate cephalad portion extending from a cephalad end of said elongate member toward said caudal portion, said elongate member further including an elongate transition portion extending between and connecting said caudal portion to said cephalad portion, wherein said caudal portion includes a cross-section orthogonal to said longitudinal axis that is elliptical and said cephalad portion includes a cross-section orthogonal to a center axis of said cephalad portion that is circular, said transition portion including a cross-section orthogonal to a center axis of said transition portion that gradually transition from said elliptical cross-section of said caudal portion at a first junction with said caudal portion to said circular cross-section at a second junction with said cephalad portion.

2. The system of claim 1, wherein when engaged to the spinal column said elongate member is configured so that a major dimension of said elliptical cross-section extends in a medial-lateral direction and a minor dimension of said elliptical cross-section extends in an anterior-posterior direction.

3. The system of claim 1, wherein said elliptical cross-section extends from said caudal end to said first and junction and said elliptical cross-section includes linear anterior and posterior sides and curved medial and lateral sides extending between and connecting said linear anterior and posterior sides.

4. The system of claim 3, wherein said linear anterior side and said linear posterior side each include a first width at said first junction, and said transition portion includes a linear posterior side and a linear anterior side each defining a width that longitudinally transition from said first width at said first junction to a point at said second junction.

5. The system of claim 4, wherein said transition portion extends from said caudal end to said cephalad end along an arc that is curved from said first junction to said second junction.

6. The system of claim 5, wherein said caudal portion includes a first length from said caudal end to said first junction, said transition portion includes a second length between said first and second junctions, and said cephalad portion includes a third length from said second junction to said cephalad end, wherein said first length is greater than said second length, and said second length is greater than said first length.

7. The system of claim 6, wherein said first length is substantially equally to said first and second lengths combined, and said second length is about twice as long as said third length.

8. The system of claim 1, wherein said elongate member further comprises a second elongate portion removably engaged to said caudal end of said caudal portion, said second elongate portion extending caudally from said caudal end, wherein said second elongate portion includes a circular cross-section extending from said caudal end.

9. The system of claim 8, wherein said at least one anchor includes a first anchor engaged to said cephalad portion and a second anchor engaged to said second elongate portion, and said caudal portion and said transition portion extend between said first and second anchors with no anchors engaged directly to said caudal portion and said transition portion.

10. The system of claim 1, wherein said caudal portion includes an inner channel extending therein from said caudal end and a slot extending through said caudal portion that opens into said channel, and further comprising:
a second elongate portion removably engaged in said channel of said caudal portion and extending caudally from said caudal end; and
a securing member around said caudal portion clampingly engaging said second elongate member in said caudal portion.

11. A spinal stabilization system, comprising:
at least one bone anchor; and
an elongate member engageable to said at least one bone anchor when said elongate member is positioned along a spinal column of a patient with a longitudinal axis of said elongate member oriented in a caudal to cephalad direction relative to the patient, wherein said elongate member includes an elongate caudal portion extending from a caudal end of said elongate member and an elongate cephalad portion extending from a cephalad end of said elongate member toward said caudal portion, said elongate member further including an elongate transition portion extending between and connecting said caudal portion to said cephalad portion, wherein said caudal portion includes a cross-section orthogonal to said longitudinal axis that is substantially stiffer between medial and lateral sides of said caudal portion than between anterior and posterior sides of said caudal portion, said cephalad portion including a cross-section orthogonal to a center axis of said cephalad portion that provides substantially the same stiffness in all directions from said center axis of said cephalad portion, and said transition portion includes a cross-section orthogonal to a center axis of said transition portion that longitudinally transitions from said cross-section of said caudal portion at a first junction with said caudal portion to said cross-section of said cephalad portion at a second junction with said cephalad portion.

12. The system of claim 11, wherein:
said cross-section of said caudal portion is elliptical from said caudal end to said first junction and said cross-section of said cephalad portion is circular from said second junction to said cephalad end; and
when engaged to the spinal column said elongate member is configured so that a major dimension of said elliptical cross-section extends in a medial-lateral direction and a minor dimension of said elliptical cross-section extends in an anterior-posterior direction.

13. The system of claim 12, wherein:
said elliptical cross-section includes linear anterior and posterior sides and curved medial and lateral sides extending between and connecting said linear anterior and posterior sides;
said linear anterior side and said linear posterior side each include a first width at said first junction, and said transition portion includes a linear posterior side and a linear anterior side each defining a width that gradually transitions from said first width at said first junction to a point at said second junction; and
said transition portion extends from said caudal end to said cephalad end along an arc that is curved from said first junction to said second junction.

14. The system of claim 11, wherein:
said caudal portion includes a first length from said caudal end to said first junction;
said transition portion includes a second length between said first and second junctions;
said cephalad portion includes a third length from said second junction to said cephalad end; and
said first length is greater than said second length, and said second length is greater than said first length with said first length substantially equally to said first and second lengths combined, and said second length is about twice as long as said third length.

15. The system of claim 11, wherein said caudal portion includes an inner channel extending therein from said caudal end and a slot extending through said caudal portion that opens into said channel, and further comprising:
a second elongate portion removably engaged in said channel of said caudal portion and extending caudally from said caudal end; and
a securing member around said caudal portion clampingly engaging said second elongate member in said caudal portion.

16. A method for spinal stabilization, comprising:
providing an elongate member for positioning along a plurality of vertebral levels of a spinal column of a patient, wherein the elongate member includes an elongate caudal portion extending from a caudal end toward a cephalad end of the elongate member, the elongate member further including a cephalad portion extending from the cephalad end toward the caudal end, and a transition portion extending from the caudal portion to the cephalad portion;
positioning the caudal portion along at least two vertebral levels of the plurality of vertebral levels with a major dimension of an elliptical cross-section of the caudal portion oriented in a medial-lateral direction to prevent lateral bending of the at least two vertebral levels and a minor dimension of the elliptical cross-section of the caudal portion oriented in the anterior-posterior direction to permit flexion and extension movement of the at least two vertebral levels along which the caudal portion extends;
positioning the cephalad portion along at least one vertebral level of the plurality of vertebral levels, wherein the cephalad portion includes a circular cross-section;
positioning the transition portion along at least two vertebral levels of the plurality of vertebral levels, wherein the transition portion includes a variable cross-section that longitudinally transitions from the elliptical cross-section of the caudal portion to the circular cross-section of the cephalad portion; and
anchoring the cephalad portion to at least one vertebra of the spinal column.

17. The method of claim 16, further comprising:
providing a second elongate member extending caudally from the caudal end of the caudal portion; and
anchoring the second elongate member to at least one vertebra of the spinal column, wherein the caudal portion and the transition portion span the vertebral levels between the anchoring of the second elongate member and the cephalad portion without being anchored to any of the vertebrae between the vertebrae to which the cephalad portion and second elongate member are anchored.

18. The method of claim 17, wherein the transition portion extends along a curve so that the cephalad end is offset anteriorly from the caudal end of the elongate member.
